SEQUENCE LISTING (8 sequences)

SEQ ID NO: 1 - DNA, 288 bp, Human immunodeficiency virus
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagac

SEQ ID NO: 2 - protein, 99 aa, Human immunodeficiency virus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKE

SEQ ID NO: 3 - DNA, 396 bp, Human immunodeficiency virus
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcagtcaggtcagccaaaattac

SEQ ID NO: 4 - protein, 135 aa, Human immunodeficiency virus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKETKEALDKIEEEQNKSKKKAQQAAADTGHSSQVSQNY

SEQ ID NO: 5 - DNA, 27 bp, ARTIFICIAL SEQUENCE
  AT96BamHI primer
taaggatccatgggtgcgagagcgtca

SEQ ID NO: 6 - DNA, 29 bp, ARTIFICIAL SEQUENCE
  AT96EcoRI primer
cgcgaatcctcagtcttttatctctatcc

SEQ ID NO: 7 - protein, 20 aa, Human immunodeficiency virus
SGGELDRWEKIRLRPGGKKK

SEQ ID NO: 8 - protein, 18 aa, Human immunodeficiency virus
CQQAAADTGHSSQVSQNY

(19) United States
(12) Patent Application Publication
(10) Pub. No.: US 2010/0310592 A1
(43) Pub. Date: Dec. 9, 2010

(54) A TRUNCATED FORM OF THE HIV P17 PROTEIN

(75) Inventor: Gianfranco Merizzi, Torino (IT)

Correspondence Address: Steinfl & Bruno, 301 N Lake Ave Ste 810, Pasadena, CA, 91101, US

(73) Assignee: Medestea Research & Production S.P.A, Collereto Giacosa (Torino) (IT)

(21) Appl. No.: 12/812,747

(22) PCT Filed: Dec. 16, 2008
(86) PCT No.: PCT/IB08/55341

§ 371 Date: Jul. 13, 2010

(30) Foreign Application Priority Data
Jan. 15, 2008 (IT) TO2008A000027

(51) Int. Cl. A61K 39/12; A61P 31/18; A61K 38/16; C07K 14/16; C07H 21/04

(52) U.S. Cl. 424/186.1; 514/3.8; 514/21.3; 530/300; 536/23.72

(57) ABSTRACT

The invention relates to a truncated form of the HIV-1 p17 protein, designated as AT96, which consists of the residues from 1 to 96 of the full-length p17 protein, as well as the corresponding nucleic acid sequences. The truncated AT96 protein has the same immunological features as the full-length p17 protein but at the same time is devoid of the typical detrimental biological activities of the HIV-1 p17 protein.

The present invention generally falls within the immunology field and in particular relates to a truncated form of the HIV p17 protein that is particularly suited to be used as a vaccine against the HIV-1 virus.

The development of an effective anti-HIV-1 vaccine represents one of the main objectives for the worldwide scientific community. Such a vaccine must primarily be able to evoke both a significant cell response and the development of a vigorous humoral activity, the latter characterized by the production of antibodies capable of neutralizing the infectivity of the virus.

In these last years, the attention of the scientific world has been directed to virus-derived polypeptides to be used as a basis for the design of antiviral vaccines.

The p17 protein, which composes the matrix of the HIV-1 virus, represents a biological target that is particularly interesting from such a point of view. In fact, even though it has been classified as a structural protein, it is now clear that p17 performs several functions in many phases of the viral replication cycle, from the entry of the virus into the cytoplasm to the integration of the viral genetic material into the cellular one up to the assembly of new virus particles, thus playing a role of primary importance not only in the virus architecture but also in the HIV-1 replication cycle.

Recent studies have enabled to point out that p17 has a structure very similar to that of gamma-interferon, a human pro-inflammatory cytokine. Moreover, cell biology studies have enabled to point out the ability of p17 to modulate the biological activity of several immune system cells, both from the native and adaptive compartments. In particular, the activity of p17 on the CD4⁺ T lymphocyte subpopulation, the preferential target for HIV-1, has showed that this viral protein is able to stimulate the lymphocyte cells by activating them and thus making them more susceptible to viral infection. Furthermore, p17 is able to induce the activated T lymphocytes to release pro-inflammatory cytokines, such as gamma-interferon and tumor necrosis factor-alpha, into the cell culture microenvironment, thus generating a favorable environment for the optimum HIV-1 replication.

The activity of p17 as a “virokine” depends on the direct interaction of the protein with a specific receptor expressed on the surface of the target cells.

The evidence that p17 is able to act, from the biological point of view, on HIV-1 target cells, by affecting their functionality and inducing an increase in the HIV-1 replication activity, causes such a protein to be considered as an excellent target for the establishment of vaccination strategies against AIDS. An essential prerequisite is that p17 should be released into the cell microenvironment from HIV-1-infected cells. Actually, in vitro HIV-1-infected H9 cells release high amounts of p17 into the cell culture supernatant. Furthermore, recently it has been proven that p17 is present as a protein deposit in the lymph nodes of HIV-1 seropositive patients, also in the anti-retroviral therapy (HAART). It is interesting to note that p17 was detectable in the lymph node tissue in the absence of viral particles and/or viral genomes. This suggests that p17, as already demonstrated for other HIV structural proteins, acts on certain target cells as an exogenous protein, independently from the presence of the virus. This explains the data obtained by some researchers which pointed out a connection between the presence of high levels of anti-p17 neutralizing antibodies and a significant delay of the acquired immunodeficiency syndrome progression. As p17 is inside the glycoprotein coat in the viral architecture and as such not accessible to antibodies, such data were inexplicable before finding out that p17 is released by the infected cells.

The International patent application WO03/016337 describes short peptides isolated from HIV p17, which represent the p17 neutralizing epitope (residues from 9 to 22) and which are capable of evoking a neutralizing immune response when administered as a vaccine.

However, the use of peptides that only represent limited epitopic regions as the basis for the formulation of vaccines or as molecules capable of stimulating and directing the humoral immune response shows some drawbacks, the major one depending on the fact that peptides usually exhibit a different conformation from that showed by the full-length protein. As a result, the antibodies produced following the immunization with such peptides bring about a poor recognition of the native viral protein and therefore generate a low-efficiency humoral immune response.

The International patent application WO03/082908 describes the use of the full-length p17 protein as a vaccine in order to evoke an immune response capable of neutralizing the immunostimulating activity exerted by p17 on human cells. However, the use of the full-length protein as a vaccine shows the drawback that the administered full-length protein is capable of having the same noxious biological effects as the native viral protein.

To overcome such drawbacks, the present invention provides a truncated form of the p17 protein, designated as “AT96”, which exhibits the same immunogenic features as the full-length p17 protein, but without showing the typical noxious biological activities of the full-length protein.

The truncated AT96 protein of the invention consists of the amino acids 1-96 of the p17 protein. Preferably, AT96 is encoded by the nucleotide sequence shown as SEQ ID NO:1 in the Sequence Listing. Still more preferably, AT96 has the amino acid sequence shown as SEQ ID NO:2 in the Sequence Listing.

The truncated AT96 protein of the invention is a very promising anti-HIV therapy molecule because, as demonstrated in the experimental section below, even though it is missing the p17 carboxy-terminal end (that is the amino acids 97-132), which is important for the biological activity of the native protein, still it retains the immunogenically important epitopes, namely the epitopes capable of promoting the neutralizing cell-mediated and humoral immune response.

The truncated AT96 protein of the invention has been produced by per se known recombinant DNA methods.

In short, the AT96 protein was produced by amplifying the nucleotide sequence encoding for the amino acids 1-96 of p17 and then cloning it into the prokaryotic pGEX-4T expression vector (GE Healthcare). Such a nucleotide sequence was obtained by mutational PCR starting from the sequence encoding for the full-length p17 derived from the BH10 virus strain (NCBI accession number M15654). The nucleotide sequence of the full-length p17 used is shown in the Sequence Listing as SEQ ID NO:3 and the corresponding encoded amino acid sequence is shown as SEQ ID NO:4.

By using the specially designed mutagenic AT96BamHI and AT96ECORI primers, the following were created:
a) the BamHI restriction site upstream of the AT96 encoding sequence, which is necessary for the subsequent cloning into the prokaryotic pGEX-4T expression vector and designed to generate a recombinant clone capable of expressing AT96 in the form of a fusion protein with the Glutathione-S-Transferase (GST) enzyme encoded by the vector; and
b) the stop codon downstream of the GAC triplet encoding the amino acid 96 of p17 and the EcoRI restriction site, which is necessary for the subsequent cloning into pGEX-4T.

The sequences of the AT96BamHI and AT96ECORI primers are shown in the Sequence Listing as SEQ ID NO:5 and SEQ ID NO:6, respectively.

The amplification reaction (final volume 200 μl) was carried out by using 20 ng of a plasmid containing the nucleotide sequence of the full-length p17 as a template. The conditions used for the PCR reaction were the following: 94° C., 30 sec; 50° C., 30 sec; 72° C., 60 sec for a total of 30 cycles. The amplified product was purified by using standard protocols (Qiaquick PCR Purification Kit, Qiagen), then digested with the restriction enzymes BamHI and EcoRI and finally cloned into the multiple cloning site of the pGEX-4T plasmid, previously digested with the same restriction enzymes. The obtained construct (pGEX-AT96) was sequenced by using the Big Dye Terminator labeling (Applied Biosystems) in conjunction with analysis by sequencer and capillary chromatography (ABI PRISM® 7700) following the standard protocol.

The recombinant DNA technology was also used for the production of the AT96 protein according to per se known methods.

The pGEX-AT96 construct (20 ng) was introduced into E. coli (BL21) by electroporation and the transformed bacteria were selected on plates containing ampicillin. The presence of the AT96 insert in the selected colonies was checked by extraction, purification and restriction of the plasmid DNA.

The positive colonies were used to set up high-volume (2-4 liters) production cultures in liquid LB medium with ampicillin. The bacteria were incubated at 37° C. until reaching an optical density value corresponding to the beginning of the plateau phase (0.8 AU) and then induced to produce the protein by the addition of IPTG (isopropyl-β-thiogalactopyranoside, Sigma Aldrich) up to a final concentration of 100 mM and at the temperature of 30° C. The IPTG, by removing the block on the lacZ operon, allows the recombinant protein to be expressed in high amounts.

The AT96 protein, produced as a fusion with the GST enzyme (Glutathione-S-Transferase, the sequence of which is already inserted into the vectors of the pGEX series), was extracted by sonication of the bacterial cells and purified by using an affinity column made up by Glutathione Sepharose 4B beads (GE Healthcare), which, as being extremely related to the GST enzyme, capture the fusion protein in a selective way.

AT96 was then separated from GST by proteolytic excision with the thrombin protease (GE Healthcare). Alternatively, it may be eluted from the glutathione sepharose matrix by using a buffer containing reduced glutathione and kept in solution with the aid of chaotropic substances. The protein was finally subjected to buffer exchange, by dialyzing it against a sodium chloride solution with a 3500 kDa cut-off cellulose membrane (Pierce), and was further purified by HPLC (GE Healthcare) with an ion exchange column.

The immunogenic features of the truncated protein were checked experimentally as described in the following experimental section, which refer to the attached figures, wherein:

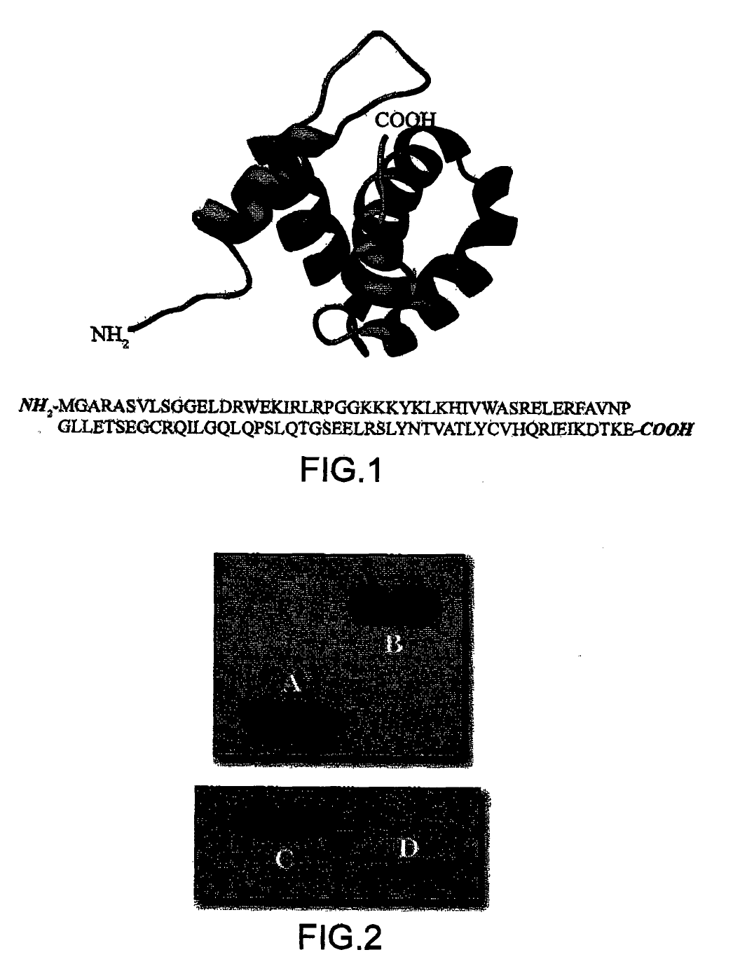

FIG. 1 represents the three-dimensional structure and the amino acid sequence (SEQ ID NO:2) of the truncated AT96 protein of the invention. The amino acid sequence given in FIG. 1 is based on the one-letter code, whereas the one given in the Sequence Listing is based on the three-letter code.

FIG. 2 illustrates the Western Blot results that aim at verifying the responsiveness of the truncated AT96 protein to anti-p17 antibodies. Following an electrophoresis run on a 15% polyacrylamide gel, the proteins were transferred onto a nitrocellulose membrane. The proteins were detected with a purified mouse monoclonal antibody (MB S-3) directed against the amino-terminal portion of p17. The membrane was developed with an HRP-conjugated anti-mouse antibody using DAB (diaminobenzidine) as the substrate. A) and C) p17 (17 KDa); B) AT96-GST (53 KDa) after elution with reduced glutathione; D) AT96 (11 KDa) after excision with the thrombin protease.

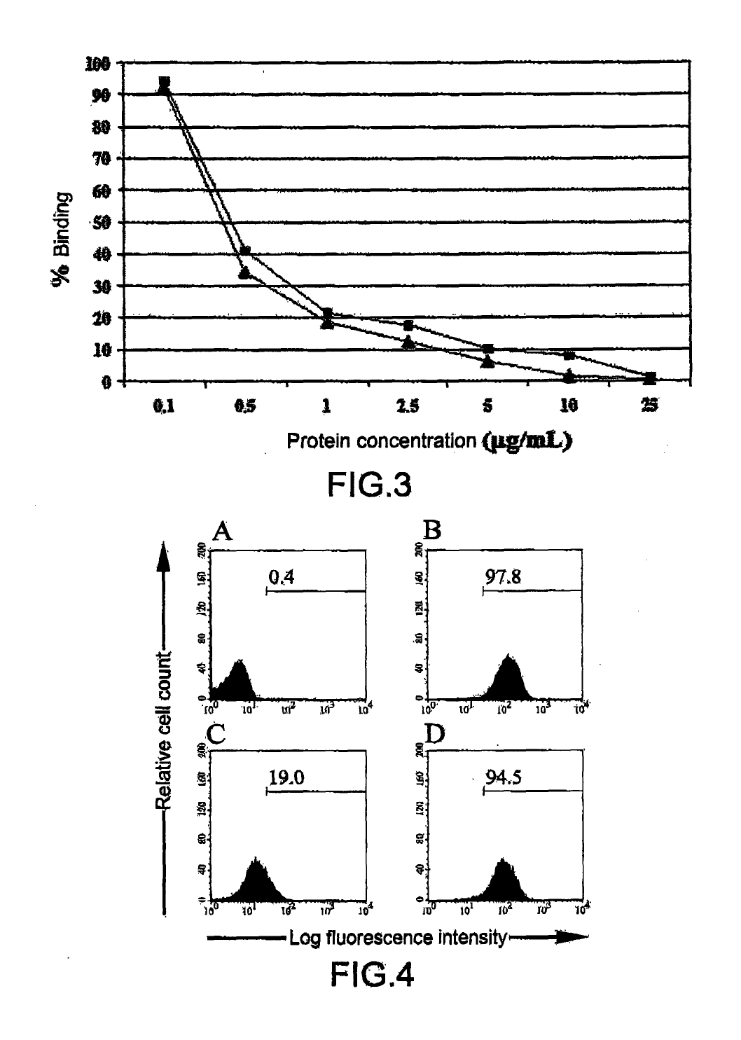

FIG. 3 shows the results of a competition ELISA test between p17 and AT96 performed to estimate the affinity of the proteins for the MBS-3 antibody. The ratio of the binding between the MBS-3 antibody and the p17 and AT96 proteins is calculated indirectly by using the immunocomplex solutions to carry out a solid-phase ELISA assay in order to detect p17. AT96 (▴) showed the same affinity for mAb as the full-length p17 (▪).

FIG. 4 shows the results from experiments concerning AT96 binding to the p17 receptor expressed on Raji cells and the neutralization of the protein-receptor interaction. A) Raji cells incubated with an unrelated protein (GST); B) Raji cells incubated with AT96 (50 ng); C) Raji cells incubated with AT96 (50 ng) in the presence of serum of a mouse immunized with a peptide capable of mimicking the amino-terminal portion of p17 (AT20) (1:50), and containing mouse neutralizing anti-p17 antibodies; D) Raji cells incubated with AT96 (50 ng) in the presence of serum of a mouse immunized with a peptide capable of mimicking the carboxy-terminal portion of p17 (CT18) (1:50).

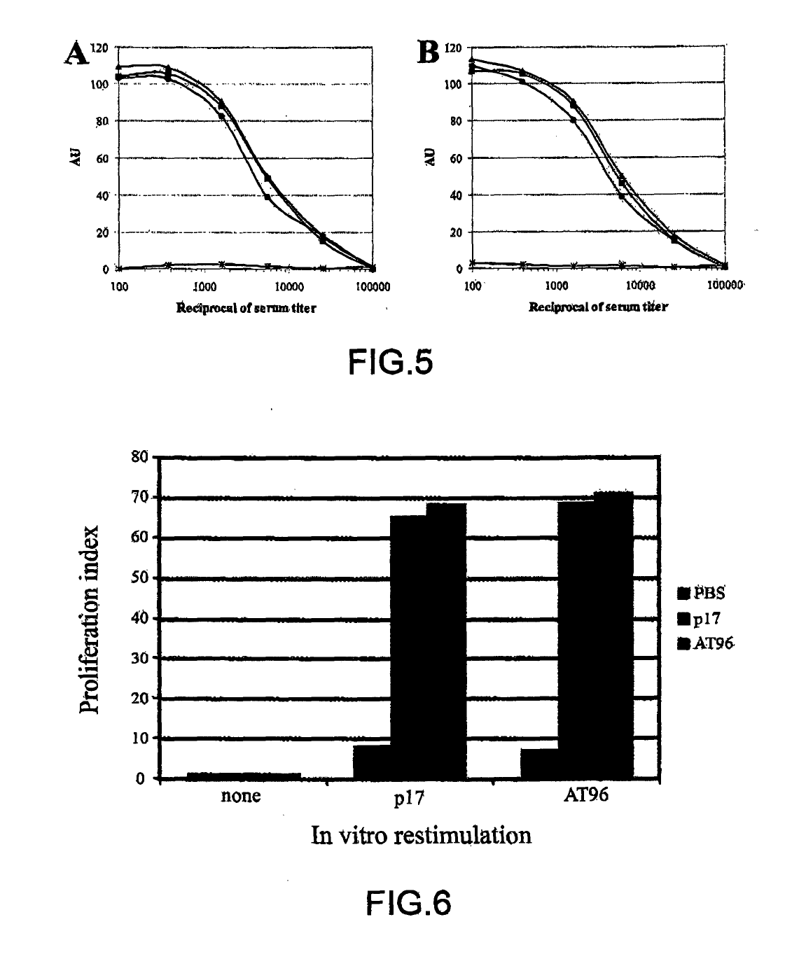

FIG. 5 shows the humoral response towards AT96 and p17 35 days after the first immunization. The antibody responses shown are from groups of mice immunized three times with (A) AT96 at the doses of 1 (), 5 (▪) and 25 (▴) μg/mouse or with p17 (B) at the doses of 1 (), 5 (▪) and 25 (▴) μg/mouse. (*) Non-immunized mice. Each serum pool was analyzed in triplicate by ELISA test.

FIG. 6 shows a comparison between the lymphoproliferative activities of p17 and AT96. Cells extracted from spleens of mice immunized with p17 or AT96 were cultured and re-stimulated for 7 days with one or the other protein to evaluate the ability of the proteins to induce clonal expansion of specific T lymphocytes. The mice were administered with 3 consecutive doses (25 μg/mouse) of p17 (middle bars), AT96 (right bars) or PBS (left bars) in combination with Freund's incomplete adjuvant. Two months after the last booster antigen injection, the mice were sacrificed and the spleens removed for the cell proliferation studies. The cells were cultured in the presence or absence of p17 or AT96. The proliferation was estimated on the basis of the incorporation of tritiated thymidine. The data are representative of 5 experiments performed with cells derived from different animals.

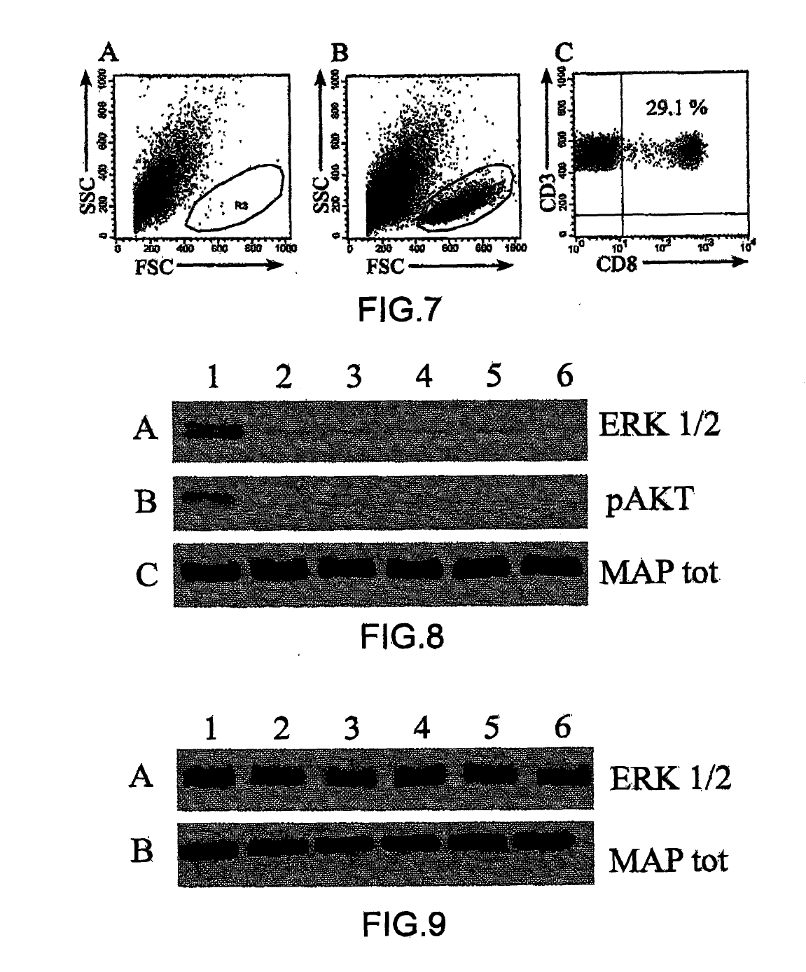

FIG. 7 shows the results relating to the specific expansion of mouse CD8⁺ T cells in response to AT96. The mice received three doses of AT96 at 25 μg/mouse with Freund's incomplete adjuvant. Two months after the last booster injection, the mice were sacrificed and the spleen cells were cultured for 7 days in the absence (A) or the presence (B) of AT96 as the booster antigen. The cells were labeled with an FITC-conjugated anti-CD8 monoclonal antibody. The cells included in the gate plotted in A and B represent, by density and size, the lymphocyte population. The data were analyzed with the CellQuest program and have been represented as a dot plot. The ratio of CD8⁺ T cells (C) in the AT96-stimulated cultures is shown in the upper right corner.

FIG. 8 relates to the assessment of the MAP kinase and pAKT phosphorylation indexes by Western Blot in Raji cells after stimulation by p17. Identification of the phosphorylated ERK1/2 (A) and pAKT (B) MAPs in Raji cells untreated with p17 (lane 1) or treated with p17 for a period of 5 minutes at the increasing concentrations of 100 ng/ml (lane 2), 200 ng/ml (lane 3), 500 ng/ml (lane 4), 1000 ng/ml (lane 5), 2000 ng/ml (lane 6). The examination of the protein amount was carried out by assessing the total MAPs (C).

FIG. 9 shows the results of the MAP kinase phosphorylation indexes obtained by Western blot assessment in Raji cells after stimulation by AT96. Identification of the phosphorylated ERK1/2 (A) MAPs in Raji cells untreated with AT96 (lane 1) or treated with AT96 for a period of 5 minutes at the increasing concentrations of 100 ng/ml (lane 2), 200 ng/ml (lane 3), 500 ng/ml (lane 4), 1000 ng/ml (lane 5), 2000 ng/ml (lane 6). The examination of the protein amount was carried out by assessing the total MAPs (B).

### EXAMPLE 1

### Recognition of AT96 by Anti-p17 Antibodies Capable of Binding to the p17 Neutralizing Epitope

The AT96 responsiveness to the anti-p17 antibodies capable of binding to the p17 neutralizing epitope was assessed by immunoenzymatic (ELISA) and Western blot assays (FIG. 2).

In order to verify that the conformation of the functional epitopes at the interaction with the cell receptor is retained in the AT96 protein, a competition ELISA test was established to evaluate the ratio of the AT96 interaction in solution with the MBS-3 monoclonal antibody, which recognizes the functional portion of p17 and inhibits the p17/receptor interaction [1]. The activity of AT96 in liquid phase as a competitor on the binding between MBS-3 and p17 present in the well (solid phase) was then compared to that of p17 used as a self-competitor.

The purified antibody was incubated with increasing concentrations of the AT96 or p17 protein and then the immunocomplexes obtained were used for ELISA assessment of the MBS-3 binding activity to the wild-type p17 fixed to the solid phase (on the bottom of the plate well). Recombinant p17, derived from the BH10 viral strain, was added to each well of a polystyrene plate for immunoenzyme assays at the concentration of 1 μg/ml in 100 μl PBS, the plates were incubated overnight at room temperature. The antigen-coated plates are designated as detection plates; whereas the uncoated plates are designated as reaction plates. In order to minimize the non-specific absorbance of the proteins, 200 μl of PBS containing 2% BSA (assay buffer) were added to each well of the detection plate. Both the reaction and detection plates were incubated for 1 hour at 37° C. The plates were washed with PBS containing 0.1% tween 20 (wash buffer). To each well of the reaction plate, 100 μl of the appropriate dilution of MBS-3 anti-p17 monoclonal antibody were added, as well as 100 μl of AT96 at concentrations ranging from 0.1 to 25 μg/ml assay buffer, or 100 μl of assay buffer alone. After a 2 hour incubation at room temperature, 100 μl aliquots were transferred from the wells of the reaction plate to those of the detection plate. The detection plates were incubated for 2 hours at room temperature and then washed four times with the wash buffer. The MBS-3 monoclonal antibody binding in solid phase was detected by adding an HRP-conjugated anti-mouse antibody.

From FIG. 3, it can be seen that the affinity of the MBS-3 antibody for AT96 is identical to that for the full-length p17 protein, confirming that the three-dimensional structure of the amino-terminal epitope recognized by the MBS-3 antibody is perfectly retained and in the truncated AT96 protein of the invention the epitope is exposed. The AT96 protein, by keeping the functional and immunogenic epitopes intact, should be able to induce, once inoculated into an organism, a significant cell-mediated and humoral anti-p17 immune response. In this context, humoral response is intended to mean the generation of antibodies that neutralize the biological activity of p17, as they are designed to block the interaction between p17 and the p17 receptor.

### EXAMPLE 2

### Interaction Between AT96 and p17 Receptor and Neutralization of the Interaction

### Materials and Methods

Cells: The ability of AT96 to bind the p17 cellular receptor was assessed on cells from the Raji lineage, that is to say Burkitt's lymphoma cells that exhibit a high expression of the p17 receptor. The cells were cultured in RPMI 1640 medium (Gibco BRL) containing 10% FCS (fetal calf serum), 100 U/ml penicillin, 50 μg streptomycin and 1 mM L-glutamine.

Conjugation of AT96 to biotin: The AT96 protein was conjugated to biotin to allow for the detection thereof by cytofluorometry through covalent binding to fluorescent streptavidin. The protein was subjected to buffer exchange through a dialysis membrane (3500 kDa cut-off, Pierce) against a sodium chloride/sodium bicarbonate solution, pH 8.0. The protein was then reacted with biotin succinimide ester (BioSPA) for 3 hours at 4° C. and further dialyzed overnight against PBS (phosphate buffered saline) containing 0.2 M sodium chloride.

Binding and neutralization tests: The ability of the sera collected from animals immunized with a peptide that mimics the amino-terminal portion of p17, designated as AT20 (SEQ ID NO: 7 in the Sequence Listing), to block AT96 binding to the p17 receptor (p17R) was assessed by cytofluorometry. The sera of AT20-immunized animals, diluted in PBS (1:50), were incubated with biotinylated AT96 (50 ng) for 20 minutes at 4° C. Raji cells were treated for 15 minutes at 4° C. with FcR blocking reagent (Miltenyi Biotech), centrifuged and then re-suspended in the sera pre-incubated with AT96. Sera from animals immunized with a peptide that mimics the carboxy-terminal portion of p17, designated as CT18, were used as a control. The cells were then washed with PBS and incubated on ice with 100 ng of streptavidin conjugated to the APC or PE Cy5.5 fluorochrome (Becton Dickinson). The cytofluorometric analysis of the samples was performed with a FACSCalibur instrument and the data were analyzed with the CellQuest Pro program (Becton Dickinson).

Synthetic peptides: The AT20 synthetic peptide consists of the 20 amino-terminal amino acids of the p17 protein, included between the amino acid positions 9 and 28 (SEQ ID NO: 7). Whereas the CT18 synthetic peptide consists of the 18 carboxy-terminal amino acids of p17, included between the amino acid positions 115 and 132 (SEQ ID NO:8). These peptides were synthesized in the free form and then were conjugated to the KLH (Keyhole Limpet Hemocyanin) carrier from Primm.

Immunizing protocol: C57BL/6 mice were immunized by the intraperitoneal route with AT20-KLH or CT18-KLH emulsified with Freund's complete adjuvant at the doses of 1, 5 and 25 μg/mouse and boosted, for 2 consecutive times at a 15 day interval, with the same dose of immunogen in incomplete adjuvant. The detection of the anti-AT20 and anti-CT18 antibodies in the sera of the immunized animals was carried out by ELISA.

ELISA: The presence of anti-AT20 and anti-CT18 antibodies in the sera of the immunized mice was assessed by an ELISA test. The wells of an ELISA plate (Nunc) were covered with 100 ng of AT96 protein overnight at room temperature in PBS. After 2 washes with the wash buffer (PBS+0.1% tween), 100 μl of the assay buffer (PBS+2% BSA) were added and incubated at 37° C. for 1 hour. After 4 washes, 100 μl of stepwise dilutions of the sera to be tested (from 1:100 to 1:3200) were transferred into each well. After incubation for 1 hour at 37° C., 4 washes were performed and 100 μl/well of a 1:1000 dilution of HRP-conjugated anti-mouse antibody were added. After incubation for 1 hour at 37° C., 4 washes were performed and 100 μl of the tetramethylbenzidine substrate were added. The chromogenic reaction was stopped with 100 μl of 2N sulphuric acid per well.

### Results

The experiments so far illustrated have allowed to establish the integrity of the amino-terminal portion of the AT96 protein, hypothesized as the region that allows p17 to interact with its cellular receptor. As a result, AT96 should be able, just like the wild-type p17 protein, to interact with the receptor on the cell membrane.

Experimentally, this has been assessed by assaying the binding of AT96 to B cells from the Raji lineage which express the p17 receptor on their surface at a high density. The cells were incubated with the biotin-conjugated AT96 protein for 30 minutes. After several washes, the binding of biotinylated AT96 to the cell surface was detected by adding fluorescent streptavidin (conjugated with the APC or PE Cy5.5 fluorochrome).

FIG. 4 shows that an unrelated biotinylated protein does not bind to Raji cells (FIG. 4A). Conversely, AT96 is able to bind the cell receptor on the membrane of Raji cells (FIG. 4B). In fact, the cytofluorometric analysis shows that the histogram in B, which represents the AT96-labeled fluorescent cells, has a higher logarithmic scale of light intensity than the histogram in A, where the cells are labeled with a molecule unable to bind the surface of Raji cells. As predicted, the binding of AT96 was detectable on the whole cell population.

Similarly, the ability of antibodies directed towards a specific amino-terminal neutralizing epitope, designated as AT20 [2], to block the in vitro interaction between p17 and the cellular receptor was also assessed. The AT96 protein was incubated with sera from animals immunized with the peptide that reproduces the sequence of the amino-terminal epitope of p17 (AT20) or with sera from animals immunized with a peptide capable of mimicking a carboxy-terminal sequence of p17 and not existing in AT96. The AT96/p17 receptor interaction was then detected by using fluorescent streptavidin as the fluorescent tracer. From the executed experiments, AT96, just like wild-type p17, resulted to be neutralized, with regard to its binding activity to the specific receptor, by antibodies directed towards the amino-terminal portion (FIG. 4C), but not by those directed towards the carboxy-terminal portion (FIG. 4D). These data confirm the hypothesis that the interaction between p17 and its receptor occurs in a highly specific way, possibly through its amino-terminal end.

### EXAMPLE 3

### Induction of Anti AT96 Immune Responses

Recently, it has been demonstrated that p17, as an antigen, is able to induce neutralizing and cell-mediated humoral immune responses in animal models [2]. Furthermore, p17 is able to act as a booster antigen in vitro, inducing proliferation of T lymphocytes taken from animals previously immunized with the viral protein.

### 3.1 Induction of the Humoral Response

Immunizing protocol: The data obtained in the present study, which was carried out by using the full-length p17 and the truncated AT96 protein of the invention in parallel, point out that the latter is also capable of inducing neutralizing humoral responses. The immunization was performed as follows: 30 9-week-old mice were immunized with different doses of p17 and AT96 administered in the presence of Freund's incomplete adjuvant. Each group, consisting of 5 mice, was administered by the intra-peritoneal route with 1, 5 and 25 μg/mouse of p17 or AT96 protein. Subsequently, 2 consecutive booster injections were given at a 15 day interval with the same dose of immunogen. The negative control was prepared by inoculating PBS and Freund's incomplete adjuvant into the mice. The immunized mice were bled at 0, 10, 24, 35 days post-immunization.

The immunization schedule with p17 and AT96 for Balb/mice is given in the following Table 1.

TABLE 1

Immunogen/mouse
(p17 or AT96 protein)

| | | | | | |
|---|---|---|---|---|---|
| Day 0 | 1st immunization | 0 μg | 1 μg | 5 μg | 25 μg |
| Day 10 | 1st bleeding | | | | |
| Day 14 | 2nd immunization | 0 μg | 1 μg | 5 μg | 25 μg |
| Day 24 | 2nd bleeding | | | | |
| Day 28 | 3rd immunization | 0 μg | 1 μg | 5 μg | 25 μg |
| Day 35 | Sacrifice | | | | |

ELISA: The sera were stored at −20° C. The serum reactivity against AT96 and p17 was tested by solid-phase ELISA, the results of which are shown in FIG. 5. The ELISA test was performed as follows. The wells of an ELISA plate (Nunc) were covered with 100 ng of AT96 protein overnight at room temperature in PBS. After 2 washes with the wash buffer (PBS+0.1% tween), 100 μl of the assay buffer (PBS+2% BSA) were added and incubated at 37° C. for 1 hour. After 4 washes, 100 μl of stepwise dilutions of the sera to be tested (from 1:100 to 1:3200) were transferred into each well. After incubation for 1 hour at 37° C., 4 washes were performed and 100 μl/well of a 1:1000 dilution of HRP-conjugated anti-mouse antibody were added. After incubation for 1 hour at 37° C., 4 washes were performed and 100 μl of the tetramethylbenzidine substrate were added. The chromogenic reaction was stopped with 100 μl of 2N sulphuric acid per well.

Neutralization test: The sera obtained through the immunizing program were used to perform neutralizing experiments on the interaction with the p17 or AT96 protein receptor. Raji cells were incubated for 30 minutes on ice with different amounts of biotinylated p17 or AT96, from 10 to 400 ng/ml. Subsequently, the cells were labeled for 30 minutes on ice with PE-conjugated streptavidin. For the neutralizing experiments, the Raji cells were incubated with the immunocomplexes obtained by pre-incubating the biotinylated p17 with sera from immunized (Ab) or non-immunized (K) animals at a 1:100 final dilution. The inhibition ratio was calculated as follows: % of receptor-positive cells in K−% of receptor-positive cells in Ab/% of receptor-positive cells in K. The Table 2 below shows the neutralization of the AT96/p17R or p17/p17R interaction by antibodies generated in animals immunized with different doses of p17. The % inhibition was calculated as indicated above.

TABLE 2

| Immunogen | | |
|---|---|---|
| (dose/mouse) | Antibody titer (no. of mice) | Percentage of inhibition |
| AT96 (25 μg) | 1:6,400-1:102,400 (n = 4) | 50-100 |
| | >1:102,400 (n = 1) | 100 |
| AT96 (5 μg) | 1:25,600-1:102,400 (n = 3) | 50-100 |
| | >1:102,400 (n = 2) | 100 |
| AT96 (1 μg) | 1:400 (n = 2) | 0 |
| | 1:25,600-1:102,400 (n = 2) | 30-70 |
| | >1:102,400 (n = 1) | 100 |
| p17 (25 μg) | 1:6.400-1:102,400 (n = 3) | 50-100 |
| | >1:102,400 (n = 2) | 100 |
| p17 (5 μg) | 1:25,600-1:102,400 (n = 3) | 50-100 |
| | >1:102,400 (n = 2) | 100 |
| p17 (1 μg) | 1:400 (n = 1) | 0 |
| | 1:25,600-1:102,400 (n = 2) | 30-70 |
| | >1:102,400 (n = 2) | 100 |

### 3.2 Induction of the Cell-Mediated Response

The AT96 protein resulted to be also able to induce, as the full-length p17, cell-mediated responses in animal models and to act in vitro as a booster antigen, inducing proliferation of T lymphocytes taken from animals immunized with the maximum dose of p17 or AT96 (FIG. 6). Similar results were obtained by stimulating spleen cells from animals immunized with the two lower doses of p17 or AT96 (data not shown).

Proliferation experiments: For the execution of the proliferation experiments, mouse spleen cells were sown into 96-well U-bottom cell culture plates at 2×10⁵ cells/well and grown in complete RPMI-1640 medium (containing penicillin, streptomycin and serum) in the presence or absence of p17 or AT96 (10 μg/ml). 5 Days later, 1 μCi of tritiated thymidine was added to the cells and these were then sacrificed after further 18 hours of culture. The data are presented as a stimulation index (SI), which is defined as the ratio of the amount of tritiated thymidine incorporated by the cells in the presence of the antigen (AT96 or p17) and the amount of tritiated thymidine incorporated by the cells in the absence of the antigen.

The cytofluorometric analyses of the spleen cell cultures extracted from animals immunized with 25 μg of AT96 and re-stimulated in vitro for seven days with the same protein, also show the specific expansion of both CD4⁺ and CD8⁺ T lymphocytes (FIG. 7). In short, the CD8⁺ lymphocytes were labeled with a specific monoclonal antibody, allowing to note that a specific expansion of both the CD4⁺ and CD8⁺ lymphocyte populations occurred in the sample boosted by the antigen (p17 or AT96). By correlating the percentage of CD8⁺ cells in the samples boosted by the antigen with the percentage in the sample lacking the booster antigen, it has been possible to calculate the CD8⁺ T cell expansion index (EI) specifically ascribable to the antigen used, p17 or AT96.

### EXAMPLE 4

### Comparison of the Biological Activity Between p17 and AT96

The biological functionality of p17 was assessed on Raji cells. First of all, the ability of p17, subsequent to its binding with the cellular receptor, to affect the activity of the cell kinases was estimated, the which cell kinases are enzymes activated through phosphorylation, which in turn are able to phosphorylate different cellular substrates, activating them. In particular, the MAPKs (ERK 1/2) and pAKT were assessed, the cell survival and the inhibition of the pro-apoptotic processes depending on the activation thereof.

The biological power of the p17 protein is demonstrated by its influence on the phosphorylation index of the kinases, also detected at low doses and after an exposition time of a few minutes. The phosphorylated kinases in the Raji cells were detected by the Western blot method using monoclonal antibodies as specific reagents (Santa Cruz). The Raji cells were stimulated for 5 minutes with different concentrations (from 25 ng/ml up to 2 μg/ml) of the p17 protein. In FIG. 8, it is possible to observe that, already at the lowest concentration, the p17 protein is able to completely inhibit the phosphorylation of both ERK 1/2 and pAKT.

Consequent to this important demonstration of the biological activity of p17 in Raji cells, we similarly proceeded to estimate the biological activity of the AT96 protein. Surprisingly, unlike what was revealed with the full-length p17, the AT96 protein proved not to be able to inhibit the phosphorylation of ERK 1/2 and pAKT, which were still active (phosphorylated) even at AT96 concentrations of 2 μg/ml (FIG. 9).

Thus, it can be inferred from the experimental data obtained that AT96 exhibits the same immunogenic features as the wild-type p17 protein, evoking both humoral and cell-mediated immune responses that are qualitatively and quantitatively similar. Also with regard to its interaction with the cellular receptor, AT96, as the wild-type p17 does, results to be able to bind the cellular receptor. Nevertheless, AT96 and the wild-type p17 differ greatly as for the cellular signal that leads to the kinase phosphorylation-mediated cell activation. Such a difference implies that the truncated AT96 protein of the invention is substantially devoid of those cell kinase activating biological activities that contribute to the activation of pro-apoptotic processes clearly noxious for the cell. On the contrary, the full-length p17 protein has such cell-damaging biological activities, which prevents the effective use thereof in anti-HIV treatment strategies.

The use of the viral truncated AT96 protein of the invention represents an extremely valuable and innovatory approach to the development of therapeutic and vaccine strategies against AIDS. Given the great importance of the matrix p17 protein in the biology of the virus, this protein represents a biological target of great interest.

The removal of the carboxy-terminal portion of p17 caused no structural alterations in the immunogenic epitopes. Moreover, the AT96 protein was shown to maintain the ability to interact with the cellular receptor.

The experimental evidences gathered during the course of this study have also allowed to extrapolate extremely significant structure-function features related to the HIV matrix p17 protein:
(i) p17 binding site for the cellular receptor is localized in the amino-terminal portion; in fact, the AT96 protein, even though it lacks the carboxy-terminal region comprised between the amino acids 97 and 132, can interact with the p17 cellular receptor;
(ii) the carboxy-terminal portion is essential to the development of the biological activity of the molecule, as AT96, unlike the wild-type p17 protein, does not inhibit the phosphorylation, hence the biological activity, of cell kinases;
(iii) the binding of p17 to the cellular receptor is not in itself indicative of biological activity. In fact, AT96 binds the receptor but does not inhibit the phosphorylation of cell kinases. It follows that the biological activity of the protein is localized in the part that has been removed, which is probably committed to interact with other functional cell structures in blocking the kinases.

On the basis of these findings, it is evident that the truncated AT96 protein of the invention may be extremely promising for the development of anti-HIV-1 vaccine strategies designed to evoke specific neutralizing, cell-mediated and humoral, immune responses without biological activities that are potentially detrimental to an HIV-1-infected organism.

Therefore, the use of the truncated AT96 protein according to the invention as a medicament, particularly as a medicament designed to evoke an immune response that neutralizes the biological activity of the HIV p17 protein, and thus suitable for use in the treatment or prevention of HIV infections, falls within the scope of the present invention.

To this end, the truncated AT96 protein of the invention may be manufactured in the form of a pharmaceutical composition, preferably an immunogenic or vaccine composition, comprising a pharmaceutically effective diluent or carrier and optionally an adjuvant. The immunogenic or vaccine pharmaceutical composition of the invention is administered through any suitable administration route including—without any limitation—the intravenous, subcutaneous, intramuscular, nasal, mucosal routes, and so on. The selection of the types and amounts of excipients, diluents, adjuvants, and carriers, which may be selected depending on the specific administration route chosen, falls within the abilities of the person of skill in the art. The immunogen dose in the pharmaceutical or vaccine composition of the invention also varies depending on several factors and the determination thereof falls within the abilities of the person of skill in the art, also taking into account the indications provided in the experimental part of the description. It is however possible to contemplate a dose comprised within the range of 1-200 μg, preferably 10-100 μg.

### BIBLIOGRAPHICAL REFERENCES

1. Papsidero L. D., Sheu M., Ruscetti F. W. Human immunodeficiency virus type 1-neutralizing monoclonal antibodies which react with p17 core protein: characterization and epitope mapping. J. Virol. 63 (1989) 267-272.
2. Fiorentini S., Marini E., Bozzo L., Trainini L., Saadoune L., Avolio M., Postillo A., Bonfanti C., Sarmientos P., Caruso A. Preclinical studies on immunogenicity of the HIV-1 p17 based synthetic peptide AT20-KLH. Biopolymers (peptide science) 76 (2004) 334-343.

1.-10. (canceled)

11. A truncated protein comprising residues 1 to 96 of a HIV-1 full-length p17 protein, the HIV-1 full-length p17 protein capable of binding a p17 receptor, the HIV-1 full-length p17 protein capable of inhibiting a cell kinase phosphorylation activity with a p17 cell kinase phosphorylation inhibition activity,
wherein
the truncated protein is capable of being recognized by an anti-p17 antibody, the anti-p17 antibody capable of binding the truncated protein with a truncated protein binding affinity, the anti-p17 antibody capable of binding the HIV-1 full-length p17 protein with a p17 protein binding affinity, the truncated protein binding affinity substantially same of the p17 protein binding affinity, the anti-p17 antibody capable of inhibiting a p17/p17 receptor interaction;
the truncated protein is capable of binding the p17 receptor;
the truncated protein is capable of evoking an anti-p17 humoral immune response, the anti-p17 humoral immune response capable of neutralizing the p17/p17 receptor interaction;
the truncated protein is capable of evoking a cell-mediated immune response in a subject to whom it is administered;
and wherein
the truncated protein substantially lacks the p17 cell kinase phosphorylation inhibition activity.

12. The truncated protein according to claim 11, wherein the P17 cell kinase phosphorylation inhibition activity is tested by assessing phosphorylation of ERK 1/2 and pAKT in Raji cells.

13. The truncated protein according to claim 11, wherein the truncated protein has the amino acid sequence of SEQ ID NO:2.

14. A medicament comprising the truncated protein according to claim 11.

15. The medicament of claim 14, wherein the medicament is capable of evoking an immune response that neutralizes a biological activity of the HIV p17 protein.

16. The medicament according to claim 15, wherein the medicament is suitable for treatment or prevention of a HIV infection.

17. A nucleic acid encoding the truncated protein according to claim 11.

18. The nucleic acid according to claim 17 having the nucleotide sequence of SEQ ID NO:1.

19. A pharmaceutical composition comprising a pharmaceutically effective amount of the truncated protein according to claim 11.

20. The pharmaceutical composition of claim 19, wherein the composition is an immunogenic or vaccine composition.

21. The pharmaceutical composition of claim 19, wherein the truncated protein has the amino acid sequence of SEQ ID NO:2.

22. A method to treat HIV in a subject, the method comprising
administering to the subject a therapeutically effective amount of the truncated protein of claim 11.

23. The method of claim 22, wherein the truncated protein has the amino acid sequence of SEQ ID NO:2.
